SEQUENCE LISTING (15 sequences)

SEQ ID NO: 1 - protein, 36 aa, Homo sapiens
MAAEPVEDNCINFVAMKFIDNTLYFIAEDDENLESD

SEQ ID NO: 2 - protein, 35 aa, Mus musculus
MAAMSEDSCVNFKEMMFIDNTLYFIPEENGDLESD

SEQ ID NO: 3 - protein, 157 aa, Homo sapiens
  MISC_FEATURE (73)..(73): Xaa is Ile or Thr
YFGKLESKLSVIRNLNDQVLFIDQGNRPLFEDMTDSDCRDNAPRTIFIISMYKDSQPRGMAVTISVKCEKISXLSCENKIISFKEMNPPDNIKDTKSDIIFFQRSVPGHDNKMQFESSSYEGYFLACEKERDLFKLILKKEDELGDRSIMFTVQNED

SEQ ID NO: 4 - protein, 193 aa, Homo sapiens
  PROPEP (1)..(36)
  CHAIN (1)..(157)
  MISC_FEATURE (109)..(109): Xaa is Ile or Thr
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISXLSCENKIISFKEMNPPDNIKDTKSDIIFFQRSVPGHDNKMQFESSSYEGYFLACEKERDLFKLILKKEDELGDRSIMFTVQNED

SEQ ID NO: 5 - protein, 157 aa, Mus musculus
  MISC_FEATURE (70)..(70): Xaa is Met or Thr
NFGRLHCTTAVIRNINDQVLFVDKRQPVFEDMTDIDQSASEPQTRLIIYMYKDSEVRGLAVTLSVKDSKXSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 6 - protein, 192 aa, Mus musculus
  PROPEP (1)..(35)
  CHAIN (1)..(157)
  MISC_FEATURE (105)..(105): Xaa is Met or Thr
MAAMSEDSCVNFKEMMFIDNTLYFIPEENGDLESDNFGRLHCTTAVIRNINDQVLFVDKRQPVFEDMTDIDQSASEPQTRLIIYMYKDSEVRGLAVTLSVKDSKXSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 7 - DNA, 582 bp, Homo sapiens
  CDS (1)..(579)
  misc_feature (73)..(73): Xaa is Ile or Thr
atggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaaytctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 8 - protein, 193 aa, Homo sapiens; identical to SEQ ID NO: 4
  misc_feature (73)..(73): The ′Xaa′ at location 73 stands for Thr, or Ile.

SEQ ID NO: 9 - DNA, 27 bp, Artificial
  Designed oligonucleotide as a sense primer for PCR to amplify a DNA fragment containing a coding sequence for a part of propeptide sequence of human IL-18 precursor
agagatctgctgctgaaccagtagaag

SEQ ID NO: 10 - DNA, 45 bp, Artificial
  Designed oligonucleotide as an antisense primer for PCR to amplify a DNA fragment containing a coding sequence for a part of propeptide sequence of human IL-18 precursor
tcaagcttagtgatggtgatggtgatgatctgattccaggttttc

SEQ ID NO: 11 - DNA, 49 bp, Artificial
  Designed oligonucleotide as a sense primer for PCR to amplify a DNA fragment containing a coding sequence for human IL-18 precursor
ggtcgggatctgtacgacgatgacgataagatggctgctgaaccagtag

SEQ ID NO: 12 - DNA, 30 bp, Artificial
  Designed oligonucleotide as an antisense primer for PCR to amplify a DNA fragment containing a coding sequence for human IL-18 precursor
tgctcgagttagtcttcgttttgaacagtg

SEQ ID NO: 13 - DNA, 47 bp, Artificial
  Designed oligonucleotide as a sense primer for PCR to amplify a DNA fragment containing a coding sequence for human IL-18 precursor
ggctagcatgactggtggacagcaaatgggtcgggatctgtacgacg

SEQ ID NO: 14 - DNA, 579 bp, Mus musculus
  CDS (1)..(576)
  misc_feature (70)..(70): Xaa is Met
atggctgccatgtcagaagactcttgcgtcaacttcaaggaaatgatgtttattgacaacacgctttactttatacctgaagaaaatggagacctggaatcagacaactttggccgacttcactgtacaaccgcagtaatacggaatataaatgaccaagttctcttcgttgacaaaagacagcctgtgttcgaggatatgactgatattgatcaaagtgccagtgaaccccagaccagactgataatatacatgtacaaagacagtgaagtaagaggactggctgtgaccctctctgtgaaggatagtaaaaygtctaccctctcctgtaagaacaagatcatttcctttgaggaaatggatccacctgaaaatattgatgatatacaaagtgatctcatattctttcagaaacgtgttccaggacacaacaagatggagtttgaatcttcactgtatgaaggacactttcttgcttgccaaaaggaagatgatgctttcaaactcattctgaaaaaaaaggatgaaaatggggataaatctgtaatgttcactctcactaacttacatcaaagttag

SEQ ID NO: 15 - protein, 192 aa, Mus musculus; identical to SEQ ID NO: 6
  misc_feature (70)..(70): The ′Xaa′ at location 70 stands for Thr, or Met.

(19) United States
(12) Patent Application Publication
(10) Pub. No.: US 2006/0073519 A1
(43) Pub. Date: Apr. 6, 2006

(54) Antibody specific to interleukin 18 precursor

(75) Inventors: Tohru Kayano, Okayama (JP); Mutsuko Taniguchi, Okayama (JP); Hiroshi Yamauchi, Okayama (JP); Masashi Kurimoto, Okayama (JP)

Correspondence Address: BROWDY AND NEIMARK, P.L.L.C.;624 NINTH STREET, NW, SUITE 300, WASHINGTON, DC, 20001-5303, US

(73) Assignee: KABUSHIKI KAISHA HAYASHIBARA SEIBUTSU KAGAKU KENKYUJO, OKAYAMA-SHI (JP)

(21) Appl. No.: 11/224,106

(22) Filed: Sep. 13, 2005

Related U.S. Application Data
(62) Division of application No. 09/711,896, filed on Nov. 15, 2000, now abandoned.

(30) Foreign Application Priority Data
Nov. 16, 1999 (JP) 324860/1999

(51) Int. Cl. G01N 33/53; A61K 39/395

(52) U.S. Cl. 435/7.1; 424/145.1

(57) ABSTRACT

Disclosed are an antibody specific to interleukin 18 (IL-18) precursor, preparation processes therefor, and uses thereof. The antibody includes immunoglobulins in both forms of polyclonal and monoclonal antibodies which exhibits an immunoreactivity against IL-18 precursor at an intensity higher than against other substances. The antibody is useful in detection and purification of IL-18 precursor and in elimination and detoxification of the precursor accumulated in vivo. The detection method using the antibody is effective in qualitative and quantitative analyses for the precursor as well as in correction of imprecise results caused by assays using anti-IL-18 antibody which exhibits a cross reactivity against the precursor.

### BACKGROUND OF THE INVENTION

1. Field of the Invention

This invention relates to a novel antibody, more particularly, an antibody specific to interleukin 18 precursor.

2. Description of the Prior Art

Interleukin 18 (hereinafter abbreviated as “IL-18”) is a type of cytokine, which mediates signal transduction in immune system. As seen in Haruki Okamura et al., Nature, Vol. 378, No. 6552, pp. 88-91 (1995), IL-18 was provisionally designated as “interferon γ inducing factor” immediately after its discovery; this designation was changed later into “IL-18” in accordance with the proposal in Shimpei Ushio et al., The Journal of Immunology, Vol. 156, pp. 4274-4279 (1996). IL-18 has a variety of biological activities, for example, inducing the production of interferon γ (hereinafter abbreviated as “IFN-γ”) in immunocompetent cells, inducing the generation of killer cells, and enhancing the cytotoxicity of killer cells, as described in these publications.

The biosynthesis of IL-18 has been well investigated, as seen in Japanese Patent Kokai No.80,270/98 by the same applicant and Kenji Akita et al., The Journal of Biological Chemistry, Vol. 272, No. 42, pp. 26595-26603 (1997). IL-18 is primarily produced in cells in the precursor form which has a molecular weight of about 24 kDa. When the precursor is exposed to the action of intracellular processing enzymes that remove N-terminal propeptide of the precursor, the precursor is converted into biologically active, mature form and then secreted from cells. The sequence listing includes examples of the amino acid sequences for human mature IL-18 and its precursor in SEQ ID NO:3 and 4, and those for murine counterparts in SEQ ID NOs:5 and 6. In SEQ ID NOs:1 and 2, the propeptide sequences of human and murine IL-18 precursors are separately shown.

Like as other cytokines, IL-18 activities are usually under strict regulation in vivo that prescribes the timings and sites to express the activities. The regulation would participate in keeping immune system and other physiological functions to work normally, and disordered expression of the activities would link with some diseases or disorders. For example, Masanori Kawashima et al. reported in Rheumatology in Europe, Journal for Education and Information in Rheumatology, vol. 26, supplement No. 2, p. 77 (1997) that IL-18 levels in body fluids were elevated specifically in patients suffering from autoimmune diseases. This report suggests a correlation between the onset of autoimmune diseases and excessive expression of IL-18 activities in vivo. The process of converting IL-18 precursor into the mature form would be involved in the regulation of expressing IL-18 activities in vivo. Therefore, it is important for diagnoses of IL-18-relating diseases to measure in vivo levels not only of mature IL-18 but also of IL-18 precursor in patients and compare the results with those in healthy specimens.

Japanese Patent Kokai Nos.217,798/96 and 231,598/96 by the same applicant disclose anti-mature-IL-18 antibodies including monoclonal antibodies and detection methods for polypeptides using the antibodies. The detection methods are useful in qualitative and quantitative analyses for mature IL-18. In contrast, there have been no established method to detect IL-18 precursor qualitatively or quantitatively; it is impossible to establish bioassays to detect the precursor, because it exhibit no activity, and there have never been disclosed any antibody specific to the precursor, feasible to detect the precursor immunologically.

The present inventors found through studying the detection methods for mature IL-18 that some anti-mature-IL-18 antibodies also exhibit an immunoreactivity against IL-18 precursor while the reaction level may be relatively low. This finding proved that detection methods using such antibodies can cause imprecise results, when employed on samples containing IL-18 together with the precursor. Methods of qualitative or quantitative analysis for IL-18 precursor were also demanded to correct the impreciseness.

### SUMMARY OF THE INVENTION

In view of the foregoing, the first object of this invention is to provide an antibody which exhibits immunoreactivity against IL-18 precursor at a high specificity.

The second object of this invention is to provide processes to prepare the antibody.

The third object of this invention is to provide uses of the antibody.

The present inventors energetically studied to attain the above objects through immunizing warm-blooded animals with IL-18 and its relating substances and comparing the antibodies produced therefrom in immunoreactivity. As a result, the inventors obtained antibodies specific to IL-18 precursor from body fluids of the animals which had been immunized with polypeptides comprising a part or whole of IL-18 precursor. Then, antibody-producing cells were collected from the animals pre-immunized similarly as above and fused with unlimitedly propagatable cells to generate hybridomas. The hybridomas thus obtained produced antibodies specific to IL-18 precursor. These antibodies were useful in various uses including the detection of IL-18 precursor. The present invention was established on the basis of these results.

In particular, the present invention attains the first object by providing an antibody specific to IL-18 precursor which is obtainable from a warm-blooded animal that has been immunized with a polypeptide comprising a part or the whole of IL-18 precursor.

The present invention attains the second object by providing a process to prepare an antibody which comprises the steps of immunizing a warm-blooded animal with a polypeptide comprising a part or the whole of IL-18 precursor and collecting an antibody specific to IL-18 precursor from a body fluid of the immunized animal, and another process which comprises the steps of culturing in vivo or in vitro an isolated cell capable of producing an antibody specific to IL-18 precursor and collecting the antibody from the resulting product of the in vivo or in vitro culture.

The present invention attains the third object by providing a detection method and purification method for IL-18 precursor using an antibody specific to IL-18 precursor and pharmaceutical uses of the antibody.

### BRIEF DESCRIPTION OF THE DRAWINGS

FIG. 1 illustrates the structure of the recombinant DNA “pRS-hproIL18”, for expression of an antigen feasible to prepare the antibody of this invention.

FIG. 2 illustrates the structure of the recombinant DNA “pRS-hproIL18”, for expression of another antigen feasible to prepare the antibody of this invention.

FIG. 3 shows the manner of selectively detecting IL-18 precursor by the detection method using the antibody of this invention “mAb-proHuIL18#75”.

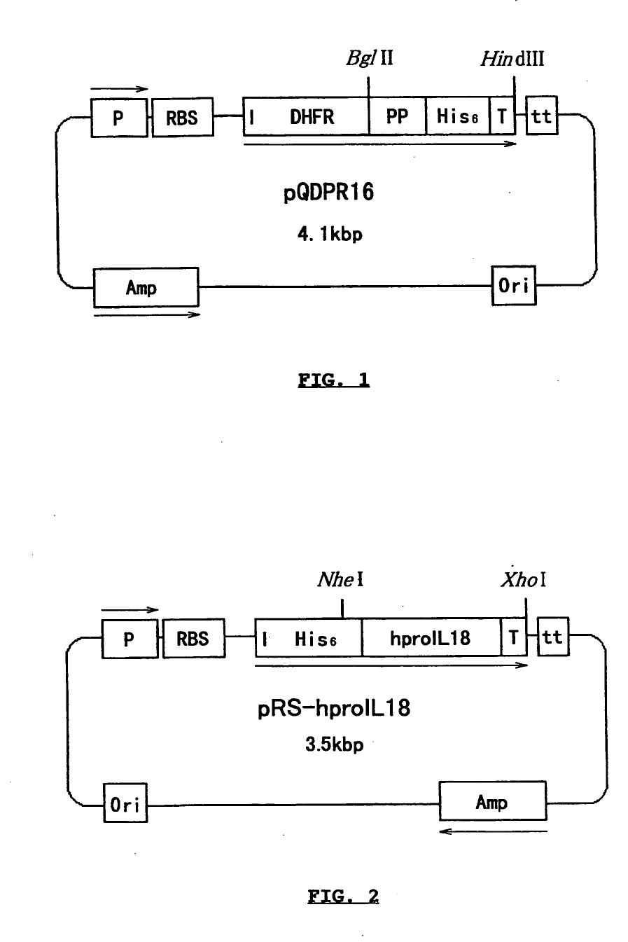

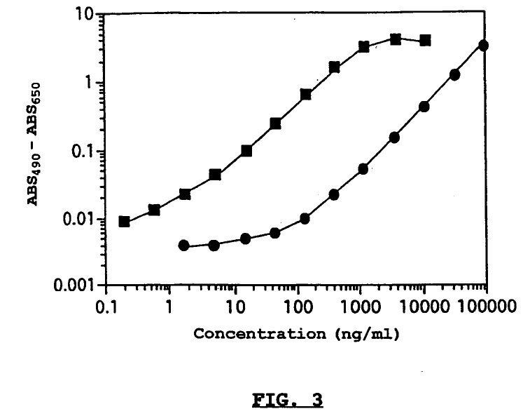

In FIGS. 1 and 2, “PP” indicates the coding region for the propeptide sequence of human IL-18 precursor; “hproIL18”, the coding region for human IL-18 precursor; “His₆”, the coding region for (His)₆ tag; “I”, initiation codon; “T”, termination codon; “DHFR”, the coding region for mouse dihydrofolate reductase; “Ori”, replication origin in Escherichia coli; “Amp”, ampicillin resistant gene; “RBS”, ribosome binding site; and “tt”, transcription termination sequence.

In FIG. 3, the axis of abscissas represents the concentration of human IL-18 precursor (solid squares) or human IL-18 (solid circles) added to well; and the axis of ordinates, the difference between absorbances at 490 nm and 650 nm of peroxidase-reacted mixture in well.

### DETAILED DESCRIPTION OF THE INVENTION

This invention relates to an antibody specific to IL-18 precursor. The term “IL-18 precursor” as used in this invention refers to the precursor proteins produced intracellularly through the process of mature IL-18 biosynthesis and structural equivalents thereof regardless of their production processes. The term “mature IL-18” as used in this invention refers to the proteins, regardless of their origins, that comprise a sequence usually consisting of 157 amino acids and capable of inducing the production of IFN-y in immunocompetent cells, inducing the generation of killer cells, and enhancing the cytotoxicity of killer cells. The sequence listing includes examples of the amino acid sequences for mature IL-18 in SEQ ID NOs:3 and 5, which are of a type of primate, human (Homo sapiens), and a type of rodent, mouse (Mus musculus), respectively (in SEQ ID NO:3, “Xaa” is threonine or isoleucine, and in SEQ ID NO:5, “Xaa” is methionine or threonine). The amino acid sequences shown in SEQ ID NOs:3 and 5 are homologous overall. These two sequences in which Xaa of SEQ ID NO:3 is threonine and Xaa of SEQ ID NO:5 is methionine show 101 identical amino acids when aligned in a usual manner for maximum matching. This means that the two sequences share 64.3% identity. The identity value indicates a feature of mature IL-18 in primary structure; mature IL-18 share usually at least 64.3%, preferably, at least 70%, and more preferably at least 80% identity with SEQ ID NO:3 or 5, calculated from the numbers of identical amino acids with SEQ ID NO:3 or 5 which are shown by conventional methods for maximum matching.

As described above, the term “IL-18 precursor” (hereinafter, occasionally designated “the precursor”) refers to the precursor proteins of IL-18 (hereinafter, the term “IL-18” alone meaning identically with “mature IL-18”), which completely contain the amino acid sequence for IL-18 but exhibiting no biological activities as observed in IL-18. In primary structure, the precursor is characterized by the N-terminal extra sequence, or so-called propeptide sequence, added to the IL-18 sequence. The sequence listing includes examples of the propeptide sequence in SEQ ID NOs:l and 2 that the precursor of human or mouse origin can contain and examples of the whole precursor sequence in SEQ ID NOs:4 and 6.

The antibody of this invention includes immunoglobulin molecules specific to the above defined IL-18 precursor in general, regardless of their origins, classes, or form (polyclonal or monoclonal antibody). The present antibody is obtainable from warm-blooded animals which have been immunized with a polypeptide comprising a part or the whole of the propeptide sequence of the precursor. The term “specific to IL-18 precursor” as used in this invention, for the characteristic of the present antibody, means that the immunoreactivity exhibited by the present antibody against IL-18 precursor is higher than that against any other substance. The term “immunoreaction” as used in this invention means the binding reaction between an antibody and a substance recognizable by the antibody, usually called antigen-antibody reaction, and the term “immunoreactivity” means the intensity of an immunoreaction. For example, when the levels of the immunoreactivity of the present antibody are compared between the cases against mature IL-18 and IL-18 precursor, the immunoreactivity against mature IL-18 is apparently lower than that against the precursor; at an intensity of, usually, at most 10%, preferably, at most 2%, and more preferably, at most 1% of that against the precursor. The levels of immunoreactivity can be compared quantitatively by conventional immunoassays, as described in detail in Examples later.

The present antibody can be easily prepared by the processes of this invention to prepare the present antibody, described below. From the processes, preferable one can be selected dependently on the objective type of the antibody.

Antigens for preparing the present antibody are not limited to those having specific structures as far as they comprise at least a part of the amino acid sequence for IL-18 precursor and can induce a desired warm-blooded animal to produce a desired antibody when administered to immunize the animal. Examples of the antigens include IL-18 precursor itself, fragments thereof containing at least a part of the propeptide sequence, and fusion proteins comprising at least one of the fragments with one or more of heterologous amino acid sequences. The wording “at least a part of the amino acid sequence for IL-18 precursor” means a partial sequence of IL-18 precursor or the propeptide sequence thereof which consists of a contiguous, usually, five or more, preferably, ten or more, and more preferably, 15 or more amino acids. The methods for preparing the antigen are not limited to specific types, and the antigen may be a recombinant or natural protein or synthesized peptide, for example. Recombinant proteins for the antigen can be obtained by conventional recombinant DNA techniques with isolated DNA molecules encoding IL-18 precursor, which are preparable from humans, mice, or others similarly as in Japanese Patent No. 2,724,987 by the same applicant. SEQ ID NOs:7 and 13 show examples of the nucleotide sequence encoding IL-18 precursor of human or mouse origin. Japanese Patent Kokai No. 80,270/98 by the same applicant discloses in detail the procedures to prepare human IL-18 precursor by recombinant DNA techniques. Fusion proteins for the antigen which comprise the propeptide sequence of IL-18 precursor and a heterologous sequence(s) can be obtained in accordance with Examples described later. IL-18 precursor proteins of animals other than humans can be obtained similarly as above with DNA molecules isolated from desired origins.

To prepare the antibody of this invention, in either form of a polyclonal or monoclonal antibody, first, warm-blooded animals can be immunized with an antigen by a conventional method. For example, an antigen can be injected alone or with an appropriate adjuvant using needles to the animals through intravenous, intracutaneous, subcutaneous, or intraperitoneal routes, and the animals can be housed for a prescribed period. The warm-blooded animals are not limited to specific species, sex, ages, or weights as far as they can produce the desired antibody. Examples of the warm-blooded animals are mammals including rodents such as mice, hamsters, rabbits, and guinea pigs and artiodactyls such as goats and sheep and birds including chickens and quails. Animals that are more suitable for immunization can be selected with respect to the origin of the antigen and the uses and objective form of the antibody, etc. The dosage of an antigen can be adjusted dependently on the species and weights of the target animals. When immunizing rodents, the total dosage is usually from 5 μg/head to 50 μg/head, which can be divided into two to 20 shots with each interval of about one to four weeks (in general, the first shot is called “primary immunization”; the shots after primary immunization, “additional immunizations”; and the final shot, “final immunization”). During and after immunization, the immunized animals can be examined for antibody productivity by conventional enzyme-immunoassay using the same antigen as in the immunization.

The present antibody in the form of a polyclonal antibody can be obtained by collecting sera (antisera) from the immunized animals through desired sites one to four weeks after immunization. The sites can be selected dependently on the species of immunized animals. The antisera can be further subjected, if necessary, to conventional methods to purify a desired class of immunoglobulin including IgG, IgA, and IgM, leading to producing the preparations of the polyclonal antibodies according to this invention at desired purification levels.

The present antibody can be also obtained from isolated cells capable of producing the present antibody. The term “isolated cells” as used in this invention refers to the cells existing apart from living bodies and capable of producing the present antibody, which include hybridomas, isolated spleen cells and lymphocytes, and transformant cells. This invention also provides such isolated cells. Any type of the isolated cells is useful to prepare the present antibody, and the hybridomas are particularly useful to prepare the monoclonal antibodies according to this invention.

To obtain the hybridomas, first, spleens can be extracted from the animals which have been immunized as above usually on day 3 to 5 after immunization and dispersed into spleen cells as antibody-producing cells. The spleen cells can be further immunized in vitro, if necessary. The spleen cells can be then fused with unlimitedly propagatable cells of a warm-blooded animal origin. The unlimitedly propagatable cells include, for example, cell lines established from mouse or rat myeloma, such as SP2/0-Ag14 (ATCC CRL-1631), P3/NSI/1-Ag4-1 (ATCC TIB-18), and P3X63Ag8 (ATCC TIB-9) and mutants thereof, from which more preferable ones can be selected with respect to compatibility to the above obtained spleen cells. To fuse the cells, desired methods can be selected from conventional ones such as those using cell-fusion accelerator including polyethylene glycol and Sendai virus or using electric pulse. The cell fusion products can be then subjected to cultivation with selection media such as HAT medium for selective propagation of fused cells, namely hybridomas. The propagated hybridomas can be examined with their culture supernatants for immunoreactivity against IL-18 precursor and IL-18. Hybridomas which exhibit desired immunoreactivity can be selected. The selected hybridomas can be cloned by conventional methods such as limiting dilution. The cloned hybridomas can be cultured in vitro or in vivo, and if necessary, the resulting cultures or body fluids can be purified by conventional methods to purify a desired class of immunoglobulin, leading to producing the preparations of the monoclonal antibodies according to this invention at desired purification levels.

Particular examples of methods conventionally used to purify antibodies include salting-out, dialysis, filtration, concentrating, centrifugation, separatory sedimentation, gel filtration chromatography, ion-exchange chromatography, affinity chromatography, high performance liquid chromatography, gel electrophoresis, and isoelectric focusing, which can be arbitrarily used for this invention, if necessary, in combination with one another. The purified preparations can be concentrated or dehydrated into the desired form of a liquid, solid, or others dependently on their uses.

The present antibody can be prepared by other methods. For example, a variety of established methods using recombinant DNA techniques can be arbitrarily applied to the preparation of the present antibody, particularly, those in the form of a monoclonal antibody. In brief, first, isolated cells capable of producing the present antibody are prepared as described above, by isolating spleen cells from pre-immunized, warm-blooded animals or establishing hybridomas or by immunizing in vitro with an antigen lymphocytes collected from warm-blooded animals including humans. Then, RNA is collected from the isolated antibody-producing cells, and DNA molecules which encode portions comprising the complementarity-determining regions of a molecule of the present antibody can be cloned from the RNA used as a template, for example, by RT-PCR method described in S. Tarran Jhones et al., Bio/technology, Vol. 9, pp. 88-89 (1991) or screening the expression library described in S. Paul, Methods in Molecular Biology, Vol. 51, pp. 355-394 (1995; Humana press Inc., Totowa, N.J., USA), which is usually called phage display library. Ligation of the cloned DNA molecules with a DNA molecule encoding a constant region of a desired immunoglobulin molecule can produce recombinant DNAs which encode polypeptides for the present antibody. Techniques for protein engineering can be conducted with the obtained recombinant DNA to alter the present antibody in primary structure as far as not substantially losing their specificity to IL-18 precursor. The present antibody by recombinant DNA techniques can be obtained by the steps of bringing the obtained recombinant DNAs into expression in desired expression systems and purifying the expressed antibodies. These recombinant techniques, combined with one another if desired, can produce not only antibodies equivalent in structure and function to those produced by warm-blooded animals other than humans but also antibodies comprising human constant regions, namely, “humanized antibodies” and “chimeric antibodies”, and antibodies substantially equivalent to those produced by human lymphocytes, namely, “human antibodies”. Tristan J. Vaughan et al. illustrate humanized, chimeric, and human antibodies and preparation methods therefor in Nature Biotechnology, Vol. 16, pp. 535-539 (1998). These methods can be arbitrarily applied to the preparation of the present antibody as above. The present antibody in the form of a chimeric, humanized, or human antibody is particularly useful in pharmaceutical uses for humans.

The present antibody, obtainable as above, can be used in a wide variety of fields where the detection of IL-18 precursor is demanded. Because IL-18 precursor usually exhibits no biological activity as observed in IL-18, no bioassay to detect the precursor is available. By using the present antibody, specific to IL-18 precursor, the detection of the precursor becomes possible. This invention also provides the detection method for IL-18 precursor, which comprises the steps of contacting the antibody with samples and detecting the precursor on the basis of the resulting immunoreaction or its intensity. The present detection method can be performed with the present antibody in accordance with, for example, conventional immunoassays which use a label to detect the immunoreactions or measure the intensities thereof. The conventional immunoassays include those using a radioactive substance, enzyme, or fluorescent substance as a label, which are called in general radioimmunoassay, enzyme-immunoassay, and fluoroimmunoassay. Enzyme-immunoassay may also be called “ELISA (enzyme-linked immunosorbent assay)”. Other examples are those using different labels, for example, metals such as colloidal gold and chelates containing metal ion such as europium ion and samarium ion. In these immunoassays, the present antibody can be labeled before use directly with a desired one or more of the labels as shown above. Alternatively, the present antibody can be labeled indirectly. For example, the present antibody is to be labeled indirectly when used in the form bound with biotin, a type of vitamin, and contacted with a label-bound avidin, a substance having affinity to biotin. Because the present antibody is specific to IL-18 precursor, such immunoassays using the present antibody can qualitatively or quantitatively detect IL-18 precursor, contained even at low levels in samples, at a desirable preciseness without wasting time by detecting or measuring the immunoreactions on the basis of the label used.

Conventional immunoassays may be performed with different two or more types of antibodies, as in so-called sandwich immunoassay. The present detection method also can be performed similarly, for example, with the monoclonal antibodies according to this invention produced by different hybridomas and/or with the polyclonal antibodies according to this invention in different preparations. Further, as described in Example 2 later, IL-18 precursor may be detected at a desirable sensitivity and selectivity by using an anti-IL-18 antibody in combination with a type of the present antibody, while the effectiveness varies dependently on the immunological properties of the antibodies used. With regard to these, the present detection method includes those which detect IL-18 precursor as far as using at least one type of the present antibody, independently of the types of other antibodies used in combination.

Available anti-IL-18 antibodies may exhibit an immunoreactivity against IL-18 precursor, whereas the reactivity may be relatively low. When immunoassays are performed with such antibodies on specimens which contain IL-18 and the precursor, the measured values would include impreciseness. The detection method of this invention makes it possible to correct the imprecise results or values caused by such immunoassays. Therefore, the detection method of this invention is effective in analyses not only for IL-18 precursor but also for IL-18.

The detection method of this invention is useful, for example: in diagnoses of diseases relating to mature IL-18 or IL-18 precursor, which may be associated with the enhancement or attenuation of IL-18 production in vivo or with disorders in the conversion of IL-18 precursor to the mature form; in analyses for mechanisms to progress such diseases; and in quality control of final and intermediate products in industrial or experimental productions of IL-18 through synthesizing IL-18 precursor. Examples of specimens analyzable by the present detection method include biological samples, in particular, those collected from living bodies such as bloods, sera, lymphs, bone marrow fluids, salivas, sweats, stools, cells, tissues, organs, and lysates of cells, tissues, and organs and those from in vitro cultures such as cultured cells and cell lines, culture supernatants thereof, and cell lysates therefrom.

The present antibody exhibits a particular efficacy to conventionally detect IL-18 precursor when incorporated in an immunoassay kit with other reagents to effect the immunoassay for IL-18 precursor, for example, another antibody and buffers, if necessary, in combination with reagents for labeling, reagents to detect the label, plates, and IL-18 precursor for standard, and if further necessary, with anti-IL-18 antibodies and IL-18. This invention also provides the kit. General procedures for immunoassay including methods for antibody preparation are described, for example, in Practical Immunoassay, edited by Wilfrid R. Butt, published by Marcel Dekker, Inc., New York, USA (1984), and in pp. 37-69 of this text, enzyme-immunoassay is particularly described. These general procedures can be arbitrarily applied to the present detection method.

The present antibody is also useful in immunoaffinity chromatography to purify IL-18 precursor. The purification method of this invention comprises the steps of contacting the present antibody with a sample containing IL-18 precursor and impurities to adsorb the precursor selectively on the antibody and desorbing the precursor from the antibody, both which are usually performed in aqueous media. For this method, the present antibody is usually used after immobilized on a water-insoluble carrier. To the antibody-immobilized carriers applying mixtures containing IL-18 precursor such as cultured transformants and partial purified preparations thereof brings the precursor to being adsorbed to the immobilized antibody in a substantially exclusive manner. The adsorbed substances can be easily desorbed by altering pH around the antibody. For example, in the case of using antibody belonging to IgG, the adsorbed substances can be desorbed from the antibody by controlling acidic conditions, usually pH 2-3; and in the case of antibody belonging to IgM, by controlling basic conditions, usually pH 10-11. By this method, IL-18 precursor can be relatively easily purified at relatively high purity.

Whereas IL-18 exhibits various activities in vivo, for example, involved in regulating immune system, IL-18 may affect body health when IL-18 activities is over or under normal ranges in vivo. This is well evidenced by the finding that suggests a correlation between the onset of autoimmune diseases and elevated IL-18 levels in vivo. It is also likely that body health is affected by the accumulation of IL-18 precursor in body fluids or cells, which may be caused by a disorder in the conversion of IL-18 precursor to the mature form. For example, accumulated IL-18 precursor may inhibit IL-18 activities, possibly resulting in disorders or diseases. The present antibody, specific to IL-18 precursor, can eliminate or detoxify the precursor accumulated in vivo. Therefore, treatments with the present antibody can remedy, alleviate, or prevent disorders or diseases caused by IL-18 precursor accumulated in vivo. In addition, IL-18 precursor may suppress the therapeutic effects of IL-18 inhibitors or neutralizers for IL-18-relating diseases when the precursor excessively exists in the patients at sites where the precursor encounters the administered inhibitors or neutralizers. Therefore, the present antibody can optimize the therapeutic effects of the inhibitors and neutralizers in such cases by blocking the suppressive actions of the precursor. With regard to these, the present antibody can be beneficially used as an effective or adjuvant ingredient for pharmaceutical compositions in general to remedy, alleviate, or prevent the diseases of humans and other animals against which the present antibody directly or indirectly effects, such as diseases relating to IL-18 precursor or IL-18. The above described, humanized, chimeric, and human antibodies according to this invention are particularly efficacious in such pharmaceutical uses for humans because the antibodies are less antigenic to humans than antibodies of any other origin. In pharmaceutical uses for animals other than humans, it is preferable to employ the present antibody of an origin corresponding to the species of animals to be treated.

This invention also provides a pharmaceutical composition comprising the present antibody, having the usefulness as above. Particular examples of diseases which can be treated with the present pharmaceutical composition include: viral infections such as hepatitis, herpes, condyloma acuminatum, and acquired immunodeficiency syndrome; bacterial infections such as candidiasis and malaria; solid malignant tumors such as epinephroma, mycosis fungoides, and chronic granulomatous disease; malignant tumors of hemocytes such as adult T-cell leukemia, acute lymphocytic leukemia, acute myelocytic leukemia, chronic myelocytic leukemia, non-T-lymphocytic leukemia, non-Hodgkin's lymphoma, multiple myeloma, and malignant lymphoma; sarcomas, sarcomatosis, sarcoids, or sarcoidosis such as leiomyosarcoma, fibrosarcoma, ophthalmic sarcoidosis, and pulmonary sarcoidosis; autoimmune diseases, allergic diseases or immune diseases such as rejection reactions against grafts, chronic graft-versus-host disease, pernicious anemia, atrophic gastritis, insulin resistant diabetes, Wegener's granulomatosis, discoid lupus erythematosus, hemophagocytic syndrome, idiopathic ulcerative colitis, cold agglutinin disease, Goodpasture's syndrome, Crohn's disease, sympathetic ophthalmia, hyperthyroidism, juvenile diabetes, Sjogren's syndrome, autoimmune hepatitis, autoimmune hemolytic anemia, myasthenia gravis, progressive systemic sclerosis, systemic lupus erythematosus, multiple cold hemoglobinuria, polymyositis, multiple nodal arteritis, idiopathic Addison's disease, idiopathic thrombocytopenic purpura, Basedow's disease, leukopenia, Behcet's disease, climacterium praecox, rheumatoid arthritis, adult Still's disease, Still's disease, rheumatopyra, chronic thyroiditis, Hodgkin's disease, DiGeorge's syndrome, acute graft-versus-host disease, HIV infection, asthma, atopic dermatitis, contact dermatitis, allergic rhinitis, pollinosis, and apitoxin allergy; hepatopathies such as viral hepatitis, alcoholic hepatitis, toxic hepatitis, primary biliary cirrhosis, fulminant hepatitis, viral cirrhosis, alcoholic cirrhosis, toxic cirrhosis, biliary cirrhosis, cholestatic hepatopathy, hepatocyte tumor, acute hepatitis, fatty liver, and hepatoma; diseases in gallbladder or biliary tract such as cholangitis, cholecystitis, primary sclerosing cholangitis, carcinoma of gallbladder, and cholangiocarcinoma; pancreatopathies such as acute pancreatitis, chronic pancreatitis, pancreatic failure, and pancreatic cysts; diseases in kidney or glomerulus such as acute nephritis, chronic renal failure, renal cancer, renal ischemia, nephrolithiasis, glomerulonephritis, glomerulitis, and glomerulosclerosis; diseases relating to circulating organs such as ischemia, congestive cardiomyopathy, cerebral ischemia, basilar artery migraine, abnormal vascularnet at brain base, cerebral apoplexy, aneurysm in basilar artery, arteriosclerosis, endangiitis, diabetes, mesenteric vascular occlusion, and superior mesenteric artery syndrome; and diseases relating to nervous system such as Parkinson's disease, myelatrophy, amyotrophic lateral sclerosis, Alzheimer's disease, dementia, cerebrovascular dementia, AIDS dementia, and encephalomyelitis. In addition, these diseases can also be diagnosed by the above described detection method of this invention.

The present pharmaceutical composition is provided in either form comprising the present antibody alone or in combination with one or more of physiologically acceptable, for example, carriers, excipients, diluents, solvents, adjuvants, and stabilizers, and if necessary, further with one or more of biologically active substances. Particular examples of the stabilizers include proteins such as serum albumen and gelatins, saccharides such as glucose, sucrose, lactose, maltose, trehalose, sorbitol, maltitol, mannitol, and lactitol, and buffers involving citrate or phosphate. Particular examples of the biological active substances include FK506, glucocbrticoid, cyclophosphamide, nitrogen mustard, triethylenethiophosphoramide, busulfan, pheniramine mustard, chlorambucil, azathioprine, 6-mercaptopurine, 6-thioguanine, 6-azaguanine, 8-azaguanine, 5-fluorouracil, cytarabine, methotrexate, aminopterin, mitomycin C, daunorubicin hydrochloride, actinomycin D, chromomycin A3, bleomycin hydrochloride, doxorubicin hydrochloride, cyclosporin A, L-asparaginase, vincristine, vinblastine, hydroxyurea, procarbazine hydrochloride, adrenocortical hormone, colloidal gold, interleukin 2, interleukin 12, interleukin 18, receptor antagonists and neutralizers for cytokines, including antibodies against interleukin 1 receptor proteins, interleukin 2 receptor proteins, interleukin 5 receptor proteins, interleukin 6 receptor proteins, interleukin 8 receptor proteins and interleukin 12 receptor proteins, respectively, and antagonists for TNF-α receptors, TNF-β receptors, interleukin 1 receptors, interleukin 5 receptors and interleukin 8 receptors. The present pharmaceutical composition contains the present antibody at a content of, usually, 0.00001% (w/w) to 100% (w/w), preferably, 0.0001% (w/w) to 20% (w/w) on the dry solid basis dependently on the species and ages of patients and the types of diseases to be treated.

The pharmaceutical composition of this invention includes pharmaceutics having a minimal unit for medication, which are in physically united formula suitable for one shot of administration and containing the present antibody at multiples (up to fourfold) or divisor (not less than 1/40) of a prescribed dose. Particular examples of the formulas of the present pharmaceutical composition include injections, liquids, powders, granules, syrups, tablets, capsules, and external agents, etc. The doses of the present pharmaceutical composition can be adjusted dependently on the species and ages of patients and the types of diseases to be treated. For example, to treat adult humans, the present pharmaceutical composition can be administered at a dose, per shot per body on the dry solid basis of the present antibody, in the range of, usually, about 0.1 μg to about 1 g, preferably, about 1 μg to about 100 mg, with a frequency of 1 to 4 shots a-day or 1 to 30 shots a week for a period over one day to one year, while the symptoms can be monitored. The administration routes are not specifically limited as far as leading to desired remedial, alleviative, or preventive effects, and the routes can be selected from, for example, oral routes and paraoral routes such as intradermal, subcutaneous, intramuscular, and intravenous routes dependently on the diseases to be treated or the formulas of the composition to be administered.

The present antibody may exhibit a cross reactivity, for example, against substances similar but not identical to IL-18 precursor in whole structure or sharing a partial structure with the precursor. The antibody of this invention exhibiting such cross reactivity is feasible in screening for IL-18-precursor-relating substances. In the screening, for example, biological samples such as cells, culture supernatants of cells, cell lysates, bloods, sera, tissues, tissue lysates, etc., and samples of peptide having at random sequences consisting of several to several tens of amino acids can be examined for immunoreactivity with the present antibody. Therefore, the present antibody can be beneficially used as a reagent to screen for the substances structurally or immunologically relating to IL-18 precursor. The substances obtainable through the screening, including small molecules such as peptides, may participate in the expression of IL-18 activities by acting as its agonist, antagonist, inhibitor, or neutralizer, so that the substances could be used as an IL-18-regulator or agent for diseases relating to IL-18. In addition, analyzing and comparing the substances in structure and function can contribute to molecular designing for further different IL-18-regulators.

The followings are particular examples of this invention. This invention is not limited to these examples, because the examples can be variously modified with regard to the technical levels in this field.

### EXAMPLE 1

Antibody Specific to IL-18 Precursor

### Example 1-1

Preparation of Antigen

A fusion protein comprising a part of the propeptide sequence of human IL-18 precursor shown in SEQ ID NO:1 linked with mouse dihydrofolate reductase (hereinafter abbreviated as “DHFR”) and (His)₆ tag was prepared by recombinant DNA techniques as follows for an antigen to prepare an antibody specific to human IL-18 precursor.

### Example 1-1(a)

Preparation of DNA Coding for Antigen

A DNA fragment encoding the propeptide sequence other than the N-terminal methionine was amplified by a conventional PCR, in which human IL-18 cDNA comprising SEQ ID NO:7 prepared as described in Japanese Patent No. 2,724,987 by the same applicant was used as a template and oligonucleotides shown in SEQ ID NOs:8 and 9 was used as sense and antisense primers, respectively. The sense primer was designed to encode N-terminus of SEQ ID NO:1 excepting the first methionine and contain the recognition site by BglII; the antisense primer, C-terminus of SEQ ID NO:1 followed by (His)₆ tag and the recognition site by HindIII. Amplified DNA fragment was cloned in a conventional cloning vector and sequenced. The DNA fragment had the desired structure.

The DNA fragment was excised from the cloning vector by digestion with BglII and HindIII and inserted into BglII-HindIII-digested “pQE-16”, an expression vector for Escherichia coli host containing mouse DHFR gene which is located under regulation of a promotor and followed by the recognition sites by BglII and HindIII, purchased from Kabushiki Kaisha QIAGEN, Tokyo, Japan. As shown in FIG. 1, in the recombinant DNA thus obtained, there existed downstream of the promotor the amino acid sequences for mouse DHFR, a part of the propeptide of human IL-18 precursor, and (His)₆ tag. Thus obtained recombinant DNA was named “pQDPR16”.

### Example 1-1(b)

Expression and Purification of Antigen

The recombinant DNA pQDPR16, obtained in Example 1-1(a), was introduced into Escherichia coli strain “JM109” by a conventional method. The transformant was pre-cultured in L broth, and 2.4 liters of 2× YT medium prepared in 500-ml-Erlenmeyer flasks at 150 ml/flask was seeded with the pre-culture at 1% by volume and incubated at 37° C. with a rotary shaker to culture the transformant, while the medium was monitored for the absorbance at 600 nm. When the absorbance reached 0.9, IPTG was added to the medium to give the final concentration of 1 mM and the incubation was further continued overnight. From the resulting culture, the cells were collected. After the cells (12.1 g by wet weight) were suspended in 480 ml of a buffer (pH 8.0) containing 8 M urea, from the suspension the expression product by the transformant was purified through Ni-NTA agarose gel column (12 ml by gel volume, QIAGEN) in accordance with manufacturer's instructions. The column-purified preparation was then dialyzed at 4° C. overnight against 10 mM Tris-HCl buffer (pH 7.2) containing 0.05% (w/v) detergent “TWEEN 20”, 5 mM 2-mercaptoethanol, 0.5 M NaCl, and 1.7 M urea. The dialyzed solution was centrifuged, and the supernatant was concentrated by ultrafiltration to give a protein concentration of about 1 mg/ml. The concentrate was centrifuged, and the supernatant was collected. The collected preparation (16 ml) contained 14.6 mg of protein, which exhibited an about 33 kDa band homogeneous on sodium dodecyl sulfate-polyacrylamide gel electrophoresis (hereinafter abbreviated as “SDS-PAGE”) in the presence of dithiothreitol (hereinafter abbreviated as “DTT”).

For control, another manipulation was conducted as above with the expression vector pQE-16 in place of the recombinant DNA pQDPR16. This manipulation produced an about 28 kDa protein homogeneous on SDS-PAGE in the presence of DTT. This result supports that the above obtained concentrate is a purified preparation of the fusion protein, comprising a part of SEQ ID NO:1 for the propeptide sequence, mouse DHFR, and (His)₆ tag. Thus an antigen was purified.

### Example 1-2

Preparation of DNA Coding for Antigen

A fusion protein comprising the whole amino acid sequence for human IL-18 precursor, shown in SEQ ID NO:4, and (His)₆ tag was prepared by recombinant DNA techniques as follows for another antigen.

### Example 1-2(a)

Preparation of DNA Coding for Antigen

To amplify a DNA fragment that contains a coding sequence for human IL-18 precursor shown in SEQ ID NO:4 and the recognition sites by NheI and XhoI added respectively to the 5′- and 3′-termini, the following PCR was conducted. A PCR mixture in the volume of 100 μl containing 1 ng of cDNA comprising the nucleotide sequence of SEQ ID NO:7, prepared as described in Japanese Patent No. 2,724,987 by the same applicant, 10 ng of the oligonucleotide shown in SEQ ID NO:10 as a sense primer, 100 ng of the oligonucleotide shown in SEQ ID NO:11 as an antisense primer, and other components as in conventional PCR at usual prescriptions was subjected to five cycles of incubation at 94° C. for 30 sec, at 53° C. for 30 sec, and at 72° C. for 30 sec. To the resulting PCR mixture, 100 ng of the oligonucleotide of SEQ ID NO:12 was added, and the mixture was subjected to three cycles of incubations at 94° C. for 30 sec, at 55° C. for 30 sec, and at 72° C. for 30 sec and then 32 cycles of incubations at 94° C. for 30 sec, at 60° C. for 30 sec, and at 72° C. for 30 sec. Amplified DNA fragment was cloned in a conventional cloning vector and sequenced. The DNA fragment had the desired structure.

The DNA fragment was excised from the cloning vector by digestion with NheI and XhoI and then inserted in a usual manner into NheI-XhoI-digested “pRSETB”, an expression vector for Escherichia Coli host which comprises a promotor-regulated coding sequence containing an initiation codon and a sequence for (His)₆ tag followed by the recognition sites by NheI and XhoI, commercialized by Invitrogen Corporation, San Diego, USA. As shown in FIG. 2, in the obtained recombinant DNA, there existed under regulation of the promotor a coding sequence for an amino acid sequence consisting of human IL-18 precursor and another sequence containing (His)₆ tag that is linked at the N-terminal of the precursor. The recombinant DNA was named “pRS-hproIL18”.

### Example 1-2(b)

Expression and Purification of Antigen

The recombinant DNA pRS-hproIL18, obtained in Example 1-2(a), was introduced into Escherichia Coli strain “BL21(DE3)pLysS” in competent cells, purchased from TOYOBO Co., Osaka, Japan. The transformant was pre-cultured in L broth, and one liter of L broth prepared in 500-ml-Erlenmeyer flasks at 150 ml/flask was seeded with the pre-culture at 1% by volume and incubated at 37° C. with a rotary shaker to culture the transformant, while the medium was monitored for absorbance at 600 nm. When the absorbance reached 0.5, IPTG was added to the medium to give a final concentration of 0.4 mM and the incubation was continued further five hours. From the resulting culture, the cells were collected. After the cells (12.1 g by wet weight) were suspended in 40 ml of 0.01 M Tris buffer (pH 8.0) containing 0.5 M urea and 0.1 M NaH₂PO₄ and sonicated, the suspension was centrifuged to precipitate the inclusion body fraction. The inclusion body fraction was washed by once repeating the treatment of suspension, sonication, and centrifugation. The washed inclusion body fraction was solubilized with 10 ml of a buffer (pH 8.0) containing 8 M urea, and from the resulting solution, the expression product by the transformant was purified through Ni-NTA agarose gel column (5 ml by gel volume, QIAGEN) in accordance with the manufacturer's instructions. The column-purified preparation was dialyzed at 4° C. overnight against 10 m Tris buffer (pH 7.2) containing 5 mM EDTA and 0.1 M NaH₂PO₄. The dialyzed solution was centrifuged to collect the supernatant, and the supernatant was concentrated by ultrafiltration to a protein concentration of about 1 mg/ml, about 4 ml in volume. The concentrate exhibited an about 30 kDa band homogenous on SDS-PAGE in the presence of DTT.

For control, another manipulation was conducted as above with the expression vector pRSETB in place of the recombinant DNA pRS-hproIL18. This manipulation did not produce the protein remarkably visible on SDS-PAGE in the presence of DTT as observed in the case with pRS-hproIL18. This supports that the above obtained concentrate is a purified preparation of the fusion protein, comprising human IL-18 precursor. Thus another antigen was purified.

### Example 1-3(a)

Immunization of mice and preparation of antibody-producing cells Eight-week-old female BALB/c mice were immunized as follows. For primary immunization, the antigen prepared in Example 1-1 was injected with needles in a usual manner in combination with complete Freund adjuvant to mice through intraperitoneal routes at 20 μg/head. Every two weeks thereafter, the antigen was injected twice to the mice for additional immunizations with incomplete Freund adjuvant through the same routes and at the same dose as the primary immunization. Bloods were collected from the mice through tail veins in a usual manner, and the sera prepared therefrom were examined for immunoreactivity against the antigen. Elevation of the desired antibody titer was observed in mice. These results mean that the sera are antisera containing polyclonal antibodies specific to human IL-18 precursor and that the polyclonal antibodies of this invention are obtainable in accordance with this Example.

The mice received additional immunizations further twice similarly as above. Two weeks thereafter, the other antigen, prepared in Example 1-2, was injected once to the mice for final immunization at 10 μg/head without adjuvant. On day 3 after the final immunization, spleens were extracted from the mice and dispersed into spleen cells as antibody producing cells.

### Example 1-3(b)

Establishment of Hybridomas

The spleen cells obtained from one mouse were suspended in serum-free RPMI1640 medium (pH 7.2, hereinafter designated “the serum-free medium”), preheated to 37° C., together with the murine bone marrow cells SP2/0-Ag14 (ATCC CRL1581) to give the respective cell densities of 1×10⁸ cells/ml and 3×10⁷ cells/ml, and the suspension was mixed well. The suspension was centrifuged, and the supernatant was removed. The cells in pellet were allowed to fuse by receiving drop by drop 1 ml of the serum-free medium containing 50% (w/v) polyethylene glycol having the mean molecular weight of 1,500 daltons over one minute and then being incubated at 37° C. for one minute. The cell fusing reaction was terminated by adding the serum-free medium to the mixture to give the final volume of 50 ml. The cell fusion product was centrifuged to separate completely from the supernatant, and the pellet was suspended in HAT medium to give a cell density of about 2×10⁵ cells/ml. The cell suspension was divided in 96-well microplates at 150 μl/well and incubated in a 5% (v/v) CO₂ incubator at 37° C. for about 10 days to let hybridomas selectively propagate.

The culture supernatants of the wells were examined for immunoreactivity against human IL-18 precursor and human IL-18 by conventional immunoassay as follows. Human IL-18 precursor, consisting of the amino acid sequence shown in SEQ ID NO:4, was prepared as described in Japanese Patent Kokai No. 80,270/98 by the same applicant and immobilized in a usual manner in the wells of plastic plates. Human IL-18, comprising the amino acid sequence shown in SEQ ID NO:3, was prepared as described in Japanese Patent No. 2,724,987 by the same applicant and immobilized as above in the wells of other plastic plates. Each well of the plates was given with a portion of any culture supernatant of the hybridomas and then washed. The wells were further given with goat anti-mouse-IgG antibody labeled with horseradish peroxidase and then washed. The wells were subjected to peroxidase reaction using o-phenylenediamine and peroxide as substrates. On the basis of the color development after the reaction, immunoreaction was judged whether occurred or not. Fifteen samples exhibited an immunoreactivity against human IL-18 precursor but not apparently against human IL-18. From the wells of the hybridoma culture that contained these samples, hybridomas were respectively collected and subjected to limiting dilution, leading to establishment of 15 independent clones of hybridomas. The clones were cultured and analyzed in a usual manner. One clone produced the IgG₂b class of monoclonal antibody; two clones, IgG₃; and the remaining 12 clones, IgM.

### Example 1-3(c)

Preparation of Monoclonal Antibodies

The hybridomas obtained as 15 independent clones in Example 1-3(b) were manipulated to prepare monoclonal antibodies as follows. Each hybridoma was suspended in RPMI1640 medium (pH 7.2) supplemented with 5%(v/v) fetal calf serum to give a cell density of 1×10⁵ cells/ml and cultured at 37° C. in a 5% (v/v) CO₂ incubator to a prescribed cell numbers. Eleven-week-old BALB/c mice received pristane at 0.5 ml/head through intraperitoneal routes. Each hybridoma was collected from the culture and injected to the pristane-injected mice at 1×10⁷ cells/head through intraperitoneal routes. The mice were housed for about one week in a usual manner.

Monoclonal antibodies from the mice that received hybridomas producing the IgG_{2b} or IgG₃ class of monoclonal antibodies were purified as follows. A column packed with the protein-A-bound gel “Protein A-Sepharose CL-4B” (Pharmacia LKB Biotechnology AB, Uppsala, Sweden) was equilibrated with 1.5 M glycine-NaOH buffer (pH 8.9) containing 3 M NaCl (hereinafter, designated “the equilibration buffer”). Ascites was collected from each housed mouse, purified by centrifugation in a usual manner, twofold diluted with the equilibration buffer, and applied to the equilibrated column. The column was washed with an adequate amount of the equilibration buffer, and then an appropriate amount of 0.1 M glycine-HCl buffer (pH 3.0) was run to elute the antibody adsorbed in the column. The eluate was recovered and dialyzed against PBS (phosphate-buffered saline) at 4° C. overnight. Thereafter the dialyzed solution was recovered. These manipulations were conducted with every hybridoma, resulting in purified preparations of three independent monoclonal antibodies: one belongs to the class IgG_{2b}; and remaining two, IgG₃.

From mice that received hybridomas producing the IgM class of monoclonal antibodies, ascites was collected, purified by centrifugation in a usual manner, and salted-out with 50%-saturated ammonium sulfate, and then the resulting precipitate was collected. The precipitate fraction was dialyzed at 4° C. overnight against 50 mM phosphate-potassium buffer (pH 6.8) containing 0.15 M NaCl. The dialyzed solution was applied to a hydroxyapatite column equilibrated with the same buffer as above, and the column was washed with an adequate amount of the same buffer. Through the column, phosphate-potassium buffer (pH 6.8) containing 0.15 M NaCl was run at the buffer concentration increasing from 50 mM to 300 mM in a linear gradient manner, and fractions eluted with the buffer at over 200 mM were collected and pooled. These manipulations were conducted with every hybridoma, resulting in purified preparations of 12 independent monoclonal antibodies which belong to the class IgM.

The monoclonal antibodies obtained in this Example were subjected to enzyme-immunoassay as described in Example 1-3(b). These monoclonal antibodies exhibited an immunoreactivity against human IL-18 precursor but not apparently against human IL-18. Thus 15 types of the present antibody were obtained. One of the monoclonal antibody belonging to the class IgG_{2b} whose immunoreactivity against IL-18 precursor was particularly high, in this assay, was named “mAb-proHuIL18#75”.

### Example 2

Enzyme-Immunoassay

Enzyme-immunoassay was conducted with two types of antibodies as follows. For the first antibody, the monoclonal antibody mAb-proHuIL18#75 was prepared in accordance with Example 1-3(c). Anti-human-IL-18 monoclonal antibodies against were prepared as described in Japanese Patent Kokai No. 231,598/96 by the same applicant. The anti-human-IL-18 monoclonal antibodies were subjected to western blotting in a usual manner. One sample, named “mAb-HuIL18#25-2G”, exhibited an immunoreactivity against human IL-18 as well as against human IL-18 precursor at an intensity comparable to that against human IL-18. For the second antibody, the monoclonal antibody mAb-HuIL18#25-2G was labeled with horseradish peroxidase in a usual manner. For test samples, human IL-18 precursor was prepared similarly as in Japanese Patent Kokai No.80,270/98 by the same applicant, and human IL-18 precursor, in accordance with Japanese Patent No.2,724,987 by the same applicant.

The first antibody was prepared in a 20 μg/ml solution with PBS and poured in microplates at. 100 μl/well. The microplates were allowed to stand at ambient temperature for three hours so that the antibody was immobilized in wells. The wells were washed with PBS, added with PBS containing 0.1% (w/v) bovine serum albumin at 200 μl/well, and allowed to stand at 4° C. for 16 hours. The wells were washed with PBS, and to each well the following materials were added: 50 μl of a solution containing human IL-18 precursor or human IL-18 prepared at any concentration shown in FIG. 3 and 50 μl of the assay buffer (PBS containing 1% (w/v) BSA, 5% (w/v) fetal calf serum, and 1 M NaCl). The microplates were gently rotated at ambient temperature for one hour. The wells were washed thrice with the washing buffer (PBS containing 0.05% (w/v) detergent “TWEEN 20”), and the second antibody, prepared and labeled above, was added to the wells at 100 μl/well. The microplates were rotated at ambient temperature for 1.5 hour. The wells were washed thrice with the washing buffer and then subjected to peroxidase reaction in a usual manner using o-phenylenediamine and peroxide as substrates. After the reaction, the reaction mixtures were examined for absorbances at 490 nm and 650 nm, and the differences between the former and latter absorbances were calculated to indicate the intensities of immunoreaction occurred in the wells.

FIG. 3 summarizes the results in the cases with human IL-18 precursor (solid squares) and those with human IL-18 (solid circles). Whereas not shown, the differences between the absorbances in the case with 0.1% (w/v) BSA in place of human IL-18 precursor or human IL-18 were under 0.01. These results mean that in this assay, a value of the difference apparently over 0.01, for example, higher than 0.05, indicates the existence of the objective protein in samples and that from the value, the concentration of the protein can be measured. With regard to this criterion, the enzyme-immunoassay of this Example proved to detect human IL-18 precursor at a concentration not less than about 5 ng/ml, as shown in FIG. 3. FIG. 3 additionally shows that this assay quantitatively detected the precursor at a concentration of about 5 ng/ml to about 100 ng/ml. On the contrary, this assay detected human IL-18 only at a concentration over about 1,000 ng/ml. These facts mean that this assay detects human IL-18 precursor selectively, about 200 times as sensitive as it does human IL-18.

The second antibody used in this Example exhibits an immunoreactivity, as mentioned above, against human IL-18 as well as against human IL-18 precursor. This means that the sensitivity of this assay that is differential to human IL-18 precursor and human IL-18 are due to the deferential immunoreactivity of the first antibody against them. Therefore, the results of this Example indicate that the first antibody is an antibody specific to human IL-18 precursor, whose immunoreactivity against human IL-18 is only at about 0.5% intensity of that against the precursor. In addition, the other monoclonal antibodies obtained in Example 1-3(c) were studied similarly as in this Example. Each antibody exhibited an immunoreactivity against human IL-18 only at about 10% to about 2% or less of that against human IL-18 precursor. Thus the antibodies were also confirmed specific to human IL-18 precursor.

Although not illustrated in detail, other antibodies specific to non-human IL-18 precursor can be prepared with reference to the disclosure by this invention. For example, SEQ ID NO:13 shows the nucleotide sequence encoding mouse IL-18 precursor, with which manipulations corresponding to Examples 1 and 2 can provide desired antibodies and detection methods. In addition, the antibody of this invention, specific to IL-18 precursor, can be beneficially used, for example, in affinity chromatography to purify IL-18 precursor and in elimination or detoxification of IL-18 precursor accumulated in vivo.

As described above, the antibody of this invention is specific to IL-18 precursor, exhibiting an immunoreactivity against IL-18 precursor at a higher intensity than against any other substance. The present antibody is feasible for various uses such as detection and purification of IL-18 precursor as well as for pharmaceutical uses. The present antibody having such usefulness can be obtained in desired amounts by the processes of this invention to prepare the present antibody.

This invention, exhibiting remarkable effects as described above, greatly contributes to the art.

While there has been described what is at present considered to be the preferred embodiments of this invention, it will be understood that the various modifications may be therein, and it is intended to cover in the appended claims all such modifications as fall within the true spirits and scope of the invention.

What is claimed is:

1. A method for treating a disease caused by an excessive accumulation of an interleukin 18 precursor comprising the amino acid sequence of SEQ ID NO:1, said method comprising administering an antibody specific to said interleukin 18 precursor to a patient in need thereof wherein said antibody does not recognize mature interleukin 18 of said interleukin 18 precursor.

2. An immunoassay method for diagnosing a disease caused by an excessive accumulation of an interleukin 18 precursor comprising the amino acid sequence of SEQ ID NO:1, said immunoassay method comprising:
i) collecting a biological sample from a human body;
ii) detecting said interleukin 18 precursor in the biological sample using an antibody specific to said interleukin 18 precursor; and
iii) evaluating the level of accumulation of said interleukin 18 precursor,
wherein said antibody specific for said interleukin 18 precursor does not recognize the mature interleukin 18 of said interleukin 18 precursor.
